(19) United States
(12) Patent Application Publication
(10) Pub. No.: US 2023/0312456 A1
(43) Pub. Date: Oct. 5, 2023

(54) VINYL ACETATE PRODUCTION PROCESS AND DEVICE

(71) Applicant: TIANJIN UNIVERSITY, Tianjin (CN)

(72) Inventors: Minhua ZHANG, Tianjin (CN); Yingzhe YU, Tianjin (CN); Hao GONG, Tianjin (CN); He DONG, Tianjin (CN); Sheng WANG, Tianjin (CN); Cheng LIU, Tianjin (CN); Xiuqin DONG, Tianjin (CN)

(21) Appl. No.: 18/023,293

(22) PCT Filed: Sep. 23, 2021
(86) PCT No.: PCT/CN2021/119873

§ 371 Date: Feb. 24, 2023

(30) Foreign Application Priority Data
Oct. 20, 2020 (CN) 2020111254261

(51) Int. Cl. C07C 69/15; B01J 7/00

(52) U.S. Cl. CPC C07C 69/15; B01J 7/00

(57) ABSTRACT

The present disclosure relates to a vinyl acetate production process and device. By arranging a stabilizing process, an acetic acid recovery system and a desorption system and device, the composition of circulating gas is changed, the explosion range is narrowed, the volume fraction of maximum permissible oxygen at an inlet of a reactor is increased under the same production load and the same catalyst condition, the safety of the production process is improved, and the conversion per pass across the reaction is increased; and meanwhile, a material separation sequence is reasonably segmented according to an actual production condition. The present disclosure arranges a first gas separating tower which recovers surplus heat of reaction gas, reduces the energy consumption of the system, and therefore the energy consumption throughout the production flow process is reduced; the present disclosure further arranges the acetic acid recovery system.

### CROSS-REFERENCE TO RELATED APPLICATIONS

This Application is a national stage application of PCT/CN2021/119873. This application claims priorities from PCT Application No. PCT/CN2021/119873, filed Sep. 23, 2021, and from the Chinese patent application 2020111254261 filed Oct. 20, 2020, the content of which are incorporated herein in the entirety by reference.

### TECHNICAL FIELD

The present disclosure belongs to the field of chemical reactions and separation, and relates to a process and device for producing vinyl acetate, in particular to a production process and device for synthesizing vinyl acetate by an ethylene gas phase method.

### BACKGROUND

Vinyl acetate, also known as VAC, has a molecular formula of CH₃COOCH=CH₂, is one of organic chemical products with the largest yield in the world, and is also one of 50 organic chemical products with the largest use amount in the world. The vinyl acetate is a simple ester of a saturated acid and an unsaturated alcohol, which can generate polyvinyl alcohol (PVA), vinyl acetate-ethylene copolymer (EVA), polyvinyl acetate (PVAC), vinyl acetate-vinyl chloride copolymer (EVC) and other polymers through self-polymerization or copolymerization with other monomers. These products are widely applied, generally can be used in adhesives, sizing agents for paper or fabrics, paint, ink, leather processing, emulsifiers, water-soluble membranes, soil conditioners, and the like, and are widely used in the fields such as chemical industry, textile, light industry, papermaking, construction and automobiles.

Generally speaking, production process routes of the vinyl acetate include an ethylene method and an acetylene method. Production by the ethylene method is predominant at present in the world. In the process of producing the vinyl acetate by the ethylene method, ethylene, oxygen and acetic acid gas, serving as raw materials, are fed into a reactor to make contact with a catalyst, and a reaction is carried out at a pressure of 0.5-1.4 MPa (G) and a temperature of 130-220° C. to generate VAC, water and a small quantity of byproducts, and high-temperature reaction gas enters a second gas separating tower after being cooled and condensed at multiple stages, so as to achieve the objective of gas-liquid separation. Unreacted ethylene gas returns to a compressor. Mixed liquid of condensed acetic acid and VAC is fed to a rectifying procedure for refining VAC.

In the process of producing the vinyl acetate by a gas phase method, the increase of the yield of the vinyl acetate is related to the concentration of oxygen in a system. By increasing the concentration of the oxygen in the reactor within a certain concentration range, the reaction temperature can be reduced, the service life of the catalyst can be prolonged, and the reaction selectivity can be improved. However, the increase of the oxygen concentration in the system is limited by an explosion limit, and the oxygen concentration is usually controlled within a small range in actual production, which will lead to the increase of the reaction temperature and the decrease of the selectivity of the vinyl acetate. As the explosion limit of the oxygen is a function of a temperature, a pressure and a mixture composition, the explosion limit of the oxygen can be changed by changing the temperature, the pressure and the mixture composition. Carbon dioxide is a byproduct generated in the process of synthesizing the vinyl acetate by the ethylene method, which can well achieve this effect. Thus, a decarburization degree is usually controlled in a decarburization procedure of an existing vinyl acetate technology, so that a certain carbon dioxide concentration is kept in the synthetic reaction gas to increase a lower explosion limit of the oxygen, thereby widening a stable area. However, since the carbon dioxide has a larger molecular weight, which makes the circulating compressor apply a large amount of work at the volume concentration at which the action of a stabilizer is achieved, the power consumption and production cost of the system are increased.

In order to prevent the decrease of the ethylene concentration after the accumulation of nitrogen and other inert gases from affecting the smooth reaction, it is necessary to extract a part of gas from the refined gas and exhaust it, and as the main composition of the exhausted refined gas is ethylene, proper absorption liquid is usually selected to recover the ethylene raw material in the exhausted gas in order to avoid waste of the ethylene raw material. The acetic acid is mainly selected as an absorbent in an existing process. In actual production, in addition to the ethylene, ethane may also be absorbed by the acetic acid, and the ethane is accumulated in production cycles, which affects the effects of emptying and impurity removal.

Patent ZL 201210385948.4 has disclosed a method for producing vinyl acetate, the invention provides a method for producing vinyl acetate by gas phase oxidation of ethylene, including an optional ethylene preparation process, a vinyl acetate synthesis process and a vinyl acetate refining process. The technological process as described in the patent neither specifies oxygen content of reaction gas, nor does it describe a stabilizing process in the specific production process, which is essential to the technological process, i.e., relates to the safety of the production process, but also directly determines the selectivity and conversion rate of the reaction.

In conclusion, the processes at the present stage mainly have the following problems:

No acetic acid recovery system is arranged, acetic acid in a tower kettle of an acetic acid evaporator is wasted or heavy components stick together in the tower kettle, resulting in the waste of the raw material and influence on the stability of actual production; the high-temperature reaction product gas exhausted from the reactor is still hot after being subjected to two-stage heat exchange with low-temperature reaction gas at an inlet of the reactor, so that heat energy is greatly wasted; a large amount of water may be generated incidentally in the process of synthesizing the vinyl acetate by the ethylene gas phase method, and this part of water is condensed by a second gas separating tower and then brought into a rectifying section to be removed, so that a large amount of steam will be consumed; and no device for recovering ethylene is arranged in the process of recovering alkaline liquid, so that the ethylene raw material is wasted along with the impurity removal of carbon dioxide in the process of recovering the alkaline liquid. The invention is not specifically designed for the stabilizing process for producing the vinyl acetate in the oxygen-containing process. Because the composition of circulating gas may determine to narrow the explosion range in the production process, the safety and production efficiency of the production process are affected. The invention is not designed for the safety of the process of producing the vinyl acetate by the ethylene gas phase method and the stabilizing process and the device therefor.

### SUMMARY

One objective of the present disclosure is to provide a vinyl acetate production process and device. By arranging a stabilizing process, an acetic acid recovery system and a desorption system and device, the composition of circulating gas is changed, the explosion range is narrowed, the volume fraction of maximum permissible oxygen at an inlet of a reactor is increased under the same production load and the same catalyst condition, the safety of the production process is improved, and the conversion per pass across the reaction is increased; and meanwhile, a material separation sequence is reasonably segmented according to an actual production condition, and a first gas separating tower is designed to recover surplus heat of reaction gas, so that the objective of pre-dehydration is achieved and the energy consumption of the systems is reduced.

In order to realize the above objective of the present disclosure, the technical solution of the present disclosure is as follows:

A production process of vinyl acetate, as shown in FIG. 1, includes a circulating gas compressor, an acetic acid evaporator, a circulating ethylene preheater, an oxygen mixer, a synthesis reactor, a first reactor outlet heat exchanger, a second reactor outlet heat exchanger, a first gas separating tower, a first gas separating tower condenser, a first gas separating tower aftercooler, a first gas separating tower phase splitter, a second gas separating tower, a degassing tank, a recovered gas compressor, a water washing tower, an absorption tower, an ethylene recovery tower, an acetic acid recovery system and a desorption system.

(1) Fresh ethylene and circulating gas are mixed, and the mixture is introduced into the circulating gas compressor for heat exchange with reaction gas by the second reactor outlet heat exchanger and then introduced into a bottom of the acetic acid evaporator. Tower bottoms of the ethylene recovery tower are sprayed from a top of the acetic acid evaporator, mixed gas of the ethylene and acetic acid is led out of the top of the evaporator, and the tower bottoms of the evaporator are fed to the acetic acid recovery system.

(2) After being led out of the top of the acetic acid evaporator, the mixed gas of the ethylene and the acetic acid is heated by the first reactor outlet heat exchanger (104) and the circulating ethylene preheater respectively and then mixed with oxygen by the oxygen mixer. The mixed gas led out of the oxygen mixer is fed into the synthesis reactor from a top.

(3) The reaction gas at an outlet of the reactor is subjected to heat exchange by the first reactor outlet heat exchanger and the second reactor outlet heat exchanger and then fed into a bottom of the first gas separating tower. Dehydrated reaction liquid is obtained at a tower kettle of the first gas separating tower and fed into a rectifying section for refining treatment. Overhead gas mainly consisting of vinyl acetate and water is obtained from a top of the first gas separating tower and fed into the first gas separating tower condenser to be condensed, non-condensible gas of the first gas separating tower condenser is fed into the first gas separating tower aftercooler to be further cooled, condensate of the first gas separating tower condenser and the first gas separating tower aftercooler enters the first gas separating tower phase splitter to be subjected to phase splitting, an oil phase obtained after phase splitting is fed into the first gas separating tower as a reflux, and a water phase is fed into the rectifying section to be further treated.

(4) Non-condensible gas cooled by the first gas separating tower aftercooler is fed into a bottom of the second gas separating tower and subjected to absorption separation by the reaction liquid and the acetic acid, then a certain amount of reaction liquid is continuously extracted from a tower kettle and fed to the degassing tank, and gas removed from the degassing tank is compressed by the recovered gas compressor and then fed into the water washing tower. Mixed gas mainly consisting of the ethylene, carbon dioxide, ethane and the oxygen is obtained on a top of the second gas separating tower and fed to the circulating gas compressor as circulating gas.

(5) After the gas fed into the water washing tower is washed with water, tower top gas is fed into the absorption tower, so that the carbon dioxide therein is absorbed by alkaline liquid, most of gas led out of a top of the absorption tower is fed to the circulating gas compressor, the rest is fed to the ethylene recovery tower and an impurity removal outlet, and tower bottoms of the absorption tower are fed into the desorption system.

(6) After the overhead gas of the absorption tower is fed into the ethylene recovery tower, fresh acetic acid is added to a top of the ethylene recovery tower to recover the ethylene gas therein, and the tower bottoms of the ethylene recovery tower are fed to the tower top of the acetic acid evaporator and the overhead gas of the ethylene recovery tower is fed to be incinerated.

In the above technical solution, the acetic acid recovery system includes an acetic acid flash tank, an acetic acid recovery tower, an acetic acid recovery tower condenser and a vacuum unit; and tower bottoms of the acetic acid evaporator firstly enter the acetic acid flash tank, gas evaporated from the flash tank is fed to the rectifying section, the tower bottoms of the flash tank are fed to the acetic acid recovery tower, overhead gas of the acetic acid recovery tower is condensed by the acetic acid recovery tower condenser and then refluxes, gas not condensed by the acetic acid recovery tower condenser is fed to the degassing tank after passing through the vacuum unit, and condensate of the vacuum unit is fed to the acetic acid recovery tower.

In the above technical solution, the desorption system includes a desorption tower and a desorption tower top condenser; and the tower bottoms of the absorption tower are fed into the desorption tower from a top of the desorption tower, two streams of materials are extracted from the top of the desorption tower, one stream is a material containing the ethylene, which is fed to the degassing tank, and the other stream mainly consists of the carbon dioxide; and after being condensed by the desorption tower top condenser, non-condensible carbon dioxide gas is fed out of a boundary area, and condensed condensate is mixed with tower bottoms of the desorption tower and fed back to the absorption tower along with added fresh alkaline liquid.

In the above technical solution, the circulating gas contains ethane gas, and a concentration of the ethane gas at an inlet of the reactor is 9-18 mol %.

In the above technical solution, an oxygen concentration at the inlet of the reactor is 6-12 mol %.

The present disclosure provides a production device of vinyl acetate, as shown in FIG. 2, including a circulating gas compressor (101), a circulating ethylene preheater (102), an acetic acid evaporator (103), a first reactor outlet heat exchanger (104), a second reactor outlet heat exchanger (105), an oxygen mixer (106), a synthesis reactor (107), a first gas separating tower (108), a first gas separating tower condenser (109), a first gas separating tower aftercooler (110), a first gas separating tower phase splitter (111), a second gas separating tower (112), a degassing tank (113), a recovered gas compressor (114), a water washing tower (115), an absorption tower (116), an ethylene recovery tower (117), an acetic acid flash tank (118), an acetic acid recovery tower (119), an acetic acid recovery tower condenser (120), a vacuum unit (121), a desorption tower (122), a desorption tower condenser (123), and a complete set of heating and conveying equipment. A connection relationship is as follows: the circulating gas compressor (101) is connected with an inlet of a heating side of the second reactor outlet heat exchanger (105); an outlet of the heating side of the second reactor outlet heat exchanger (105) is connected with an inlet in a bottom of the acetic acid evaporator (103); an outlet in a top of the acetic acid evaporator (103) is connected with an inlet of a heating side of the first reactor outlet heat exchanger (104); an outlet of the heating side of the first reactor outlet heat exchanger (104) is connected with the circulating ethylene preheater (102)); the circulating ethylene preheater (102) is connected with the oxygen mixer (106); an outlet of the oxygen mixer (106) is connected with an inlet of the synthesis reactor (107); an outlet of the synthesis reactor (107) is sequentially connected with cooling sides of the first reactor outlet heat exchanger (104) and the second reactor outlet heat exchanger (105); an outlet of the cooling side of the second reactor outlet heat exchanger (105) is connected with a feed port in a bottom of the first gas separating tower (108); a top of the first gas separating tower (108) is sequentially connected with the first gas separating tower condenser (109) and the first gas separating tower aftercooler (110); the first gas separating tower condenser (109) and the first gas separating tower aftercooler (110) are connected with the first gas separating tower phase splitter (111); a water side of the first gas separating tower phase splitter (111) is fed to a rectifying section, and an oil side is connected with a reflux port of the first gas separating tower (108); a non-condensible gas outlet of the first gas separating tower aftercooler (110) is connected with a feed port in a bottom of the second gas separating tower (112); a top of the second gas separating tower (112) is connected with the circulating gas compressor (101), and a tower kettle outlet of the second gas separating tower (112) is connected with the degassing tank (113); a gas phase outlet of the degassing tank (113) is connected with the recovered gas compressor (114); the recovered gas compressor (114) is connected with an inlet of the water washing tower (115); a tower top outlet of the water washing tower (115) is connected with an inlet in a bottom of the absorption tower (116); an outlet in a top of the absorption tower (116) is connected with the circulating gas compressor (101), an impurity removal outlet and an inlet in a bottom of the ethylene recovery tower (117), and a tower kettle outlet of the ethylene recovery tower (117) is connected with an inlet in a top of the acetic acid evaporator (103); a tower kettle outlet of the acetic acid evaporator (103) is connected with the acetic acid flash tank (118), and an outlet in a bottom of the acetic acid flash tank (118) is connected with a feed port of the acetic acid recovery tower (119); a tower top outlet of the acetic acid recovery tower (119) is connected with the acetic acid recovery tower condenser (120), a condensate outlet of the acetic acid condenser is connected with a reflux port in a tower top of the acetic acid recovery tower (119), and a non-condensible gas outlet of the acetic acid recovery tower condenser (120) is connected with the vacuum unit (121); a liquid phase outlet of the vacuum unit (121) is connected with a feed port of the acetic acid recovery tower (119), and a gas phase outlet of the vacuum unit (121) is connected with an inlet of the degassing tank (113); and a tower kettle of the absorption tower (116) is connected with a tower top inlet of the desorption tower (122), a carbon dioxide stream outlet in a top of the desorption tower (122) is connected with an inlet of the desorption tower condenser (123), and a condensate outlet of the desorption tower condenser (123) is connected with an absorption liquid feed port of the absorption tower (116).

In the above technical solution, an operating pressure of the acetic acid evaporator (103) is 1.0-1.2 bara, and a tower top temperature is 40-100° C.

In the above technical solution, a reaction temperature of the synthesis reactor (107) is 100-180° C., and a reaction pressure is 1.0-1.2 bara; an operating pressure of the first gas separating tower (108) is 6-9 bara, and a tower top temperature is 65-100° C.; and an operating pressure of the second gas separating tower (112) is 6-9 bara, and a tower top temperature is 20-50° C.

In the above technical solution, an operating pressure of the water washing tower (115) is 8-11 bara, and a tower top temperature is 22-55° C.; an operating pressure of the absorption tower (116) is 8-11 bara, and a tower top temperature is 92-112° C.; and an operating pressure of the ethylene recovery tower (117) is 7-8 bara, and a tower top temperature is 23-45° C.

In the above technical solution, an operating pressure of the acetic acid flash tank (118) is 1.0-1.2 bara, and a tower top temperature is 92-115° C.; an operating pressure of the acetic acid recovery tower (119) is 1.0-1.2 bara, and a tower top temperature is 77-91° C.; and an operating pressure of the desorption tower (122) is 1.0-1.3 bara, and a tower top temperature is 103-124° C.

As understood by those skilled in the art, vinyl acetate synthetic reaction liquid also contains acetic acid, water, low-boiling-point components and high-boiling-point components in addition to vinyl acetate. After part of a liquid mixture with a significant difference in boiling points of all components is vaporized at a certain temperature, a composition of a gas phase thereof differs from a composition of a liquid phase, and a proportion of volatile substances in the gas phase is more than that of volatile substances in the liquid phase, so that the liquid mixture can be separated and purified by a rectifying method. Generally, a vinyl acetate rectifying section (vinyl acetate refining process) includes an acetic acid tower, a coarse VAC tower, a fine VAC tower, a de-heavy component tower, an aldehydo-ester concentration tower, an acetaldehyde tower and an acetic acid recovery tower. In vinyl acetate rectifying, all components in the reaction liquid are separated through a series of rectifying operations by means of the difference in relative volatility among the components, and finally vinyl acetate products with high purity and various byproducts are obtained.

The present disclosure has the advantages and beneficial effects:
1. The composition of the circulating gas is changed, the explosion range is narrowed, and the volume fraction of the maximum permissible oxygen at the inlet of the reactor is increased under the same production load and the same catalyst condition, so that the safety of the production process is improved.
2. The oxygen concentrations at the inlet and the outlet of the reactor are increased, so that the selectivity is improved under the same time and the same catalyst condition, the production capacity is also improved accordingly, the consumption of raw materials is reduced, and the yield of products is increased.
3. The volume of the circulating gas under the same production capacity is decreased, so that the service power of the circulating compressor is reduced, the power consumption is reduced, and the production cost is reduced.
4. Heat of the reaction gas is fully recovered by arranging the first gas separating tower, the energy consumption of the production process is reduced, and therefore the energy consumption throughout the production flow process is reduced; the acetic acid recovery system is arranged, a negative-pressure environment is supplied to the acetic acid recovery tower through the vacuum unit, the acetic acid is recovered to the maximum degree, and meanwhile, accumulation of heavy components at the bottom of the acetic acid evaporator is avoided, thereby ensuring the production stability; and the device for recovering the ethylene in the process of recovering alkaline liquid is designed for the desorption tower, so that the ethylene raw material recovered from the alkaline liquid is reused.

### BRIEF DESCRIPTION OF THE DRAWINGS

FIG. 1 is a flowchart of a production process for synthesizing vinyl acetate according to the present disclosure; and

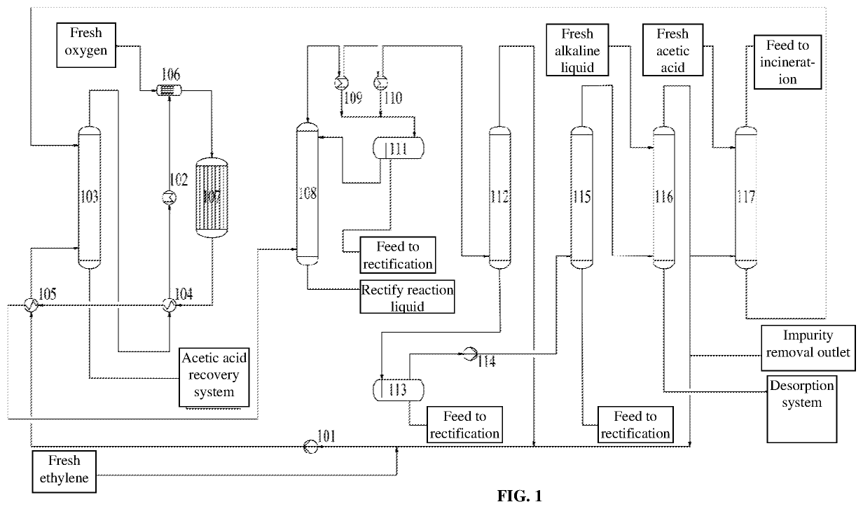

FIG. 2 is a schematic diagram of a production device and flow of vinyl acetate according to the present disclosure.

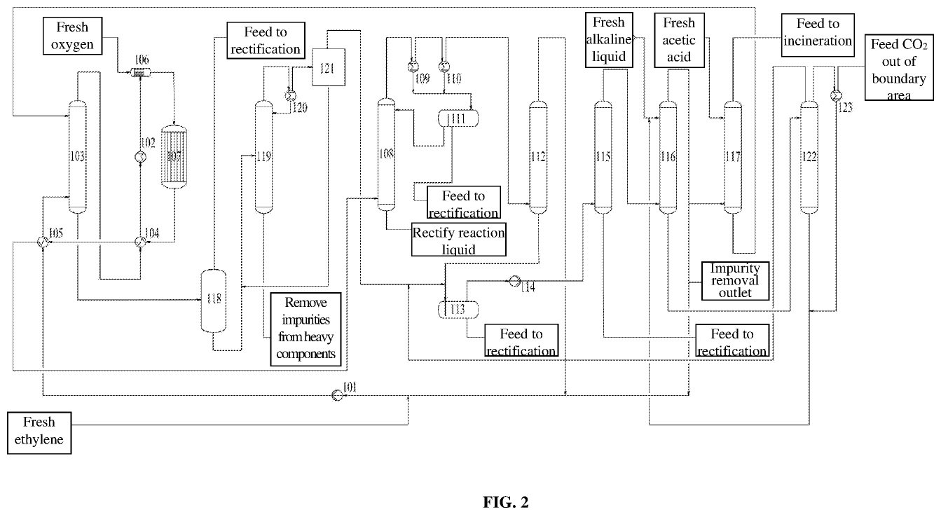

Names and numbers of circulating gas compressor (101), circulating ethylene preheater (102), acetic acid evaporator (103), first reactor outlet heat exchanger (104), second reactor outlet heat exchanger (105), oxygen mixer (106), synthesis reactor (107), first gas separating tower (108), first gas separating tower condenser (109), first gas separating tower aftercooler (110), first gas separating tower phase splitter (111), second gas separating tower (112), degassing tank (113), recovered gas compressor (114), water washing tower (115), absorption tower (116), ethylene recovery tower (117), acetic acid flash tank (118), acetic acid recovery tower (119), acetic acid recovery tower condenser (120), vacuum unit (121), desorption tower (122) and desorption tower condenser (123) are shown in the figures.

### DETAILED DESCRIPTION OF THE PRESENT DISCLOSURE

The present disclosure is further described in detail below with reference to the accompanying drawings and specific embodiments. The following embodiments are merely descriptive and not restrictive, and do not limit the scope of protection of the present disclosure.

The present disclosure provides a production process and device for synthesizing vinyl acetate, including the following technical solution:
(1) Fresh ethylene and circulating gas are mixed, and the mixture is introduced into the circulating gas compressor for heat exchange with reaction gas by the second reactor outlet heat exchanger and then introduced into a bottom of the acetic acid evaporator. Tower bottoms of the ethylene recovery tower are sprayed from a top of the acetic acid evaporator, mixed gas of the ethylene and acetic acid is led out of the top of the evaporator, and the tower bottoms of the evaporator are fed to the acetic acid recovery system.
(2) After being led out of the top of the acetic acid evaporator, the mixed gas of the ethylene and the acetic acid is heated by the first reactor outlet heat exchanger (104) and the circulating ethylene preheater respectively and then mixed with oxygen by the oxygen mixer. The mixed gas led out of the oxygen mixer is fed into the synthesis reactor from a top.
(3) The reaction gas at an outlet of the reactor is subjected to heat exchange by the first reactor outlet heat exchanger and the second reactor outlet heat exchanger and then fed into a bottom of the first gas separating tower. Dehydrated reaction liquid is obtained at a tower kettle of the first gas separating tower and fed into a rectifying section for refining treatment. Overhead gas mainly consisting of vinyl acetate and water is obtained from a top of the first gas separating tower and fed into the first gas separating tower condenser to be condensed, non-condensible gas of the first gas separating tower condenser is fed into the first gas separating tower aftercooler to be further cooled, condensate of the first gas separating tower condenser and the first gas separating tower aftercooler enters the first gas separating tower phase splitter to be subjected to phase splitting, an oil phase obtained after phase splitting is fed into the first gas separating tower as a reflux, and a water phase is fed into the rectifying section to be further treated.
(4) Non-condensible gas cooled by the first gas separating tower aftercooler is fed into a bottom of the second gas separating tower and subjected to absorption separation by the reaction liquid and the acetic acid, then a certain amount of reaction liquid is continuously extracted from a tower kettle and fed to the degassing tank, and gas removed from the degassing tank is compressed by the recovered gas compressor and then fed into the water washing tower. Mixed gas mainly consisting of the ethylene, carbon dioxide, ethane and the oxygen is obtained on a top of the second gas separating tower and fed to the circulating gas compressor as circulating gas.
(5) After the gas fed into the water washing tower is washed with water, tower top gas is fed into the absorption tower, so that the carbon dioxide therein is absorbed by alkaline liquid, most of gas led out of a top of the absorption tower is fed to the circulating gas compressor, the rest is fed to the ethylene recovery tower and an impurity removal outlet, and tower bottoms of the absorption tower are fed into the desorption system.
(6) After the overhead gas of the absorption tower is fed into the ethylene recovery tower, fresh acetic acid is added to a top of the ethylene recovery tower to recover the ethylene gas therein, and the tower bottoms of the ethylene recovery tower are fed to the tower top of the acetic acid evaporator and the overhead gas of the ethylene recovery tower is fed to be incinerated.

In the above technical solution, the acetic acid recovery system includes a primary flash tank, an acetic acid recovery tower, an acetic acid recovery tower condenser and a vacuum unit; and tower bottoms of the acetic acid evaporator firstly enter the acetic acid flash tank, gas evaporated from the flash tank is fed to the rectifying section, the tower bottoms of the flash tank are fed to the acetic acid recovery tower, overhead gas of the acetic acid recovery tower is condensed by the acetic acid recovery tower condenser and then refluxes, uncondensed gas is fed to the degassing tank after passing through the vacuum unit, and condensate of the vacuum unit is fed back to the acetic acid recovery tower.

In the above technical solution, the acetic acid recovery system includes an acetic acid flash tank, an acetic acid recovery tower, an acetic acid recovery tower condenser and a vacuum unit; and tower bottoms of the acetic acid evaporator firstly enter the acetic acid flash tank, gas evaporated from the flash tank is fed to the rectifying section, the tower bottoms of the flash tank are fed to the acetic acid recovery tower, overhead gas of the acetic acid recovery tower is condensed by the acetic acid recovery tower condenser and then refluxes, gas not condensed by the acetic acid recovery tower condenser is fed to the degassing tank after passing through the vacuum unit, and condensate of the vacuum unit is fed to the acetic acid recovery tower.

In the above technical solution, the desorption system includes a desorption tower and a desorption tower top condenser; and the tower bottoms of the absorption tower are fed into the desorption tower from a top of the desorption tower, two streams of materials are extracted from the top of the desorption tower, one stream is a material containing the ethylene, which is fed to the degassing tank, the other stream mainly consists of the carbon dioxide; and after being condensed by the desorption tower top condenser, non-condensible carbon dioxide gas is fed out of a boundary area, and condensed condensate is mixed with tower bottoms of the desorption tower and fed back to the absorption tower along with added fresh alkaline liquid.

In the above technical solution, the circulating gas contains ethane gas, and a concentration of the ethane gas at an inlet of the reactor is 9-18 mol %.

In the above technical solution, an oxygen concentration at the inlet of the reactor is 6-12 mol %.

The present disclosure provides a production device of vinyl acetate, including a circulating gas compressor (101), a circulating ethylene preheater (102), an acetic acid evaporator (103), a first reactor outlet heat exchanger (104), a second reactor outlet heat exchanger (105), an oxygen mixer (106), a synthesis reactor (107), a first gas separating tower (108), a first gas separating tower condenser (109), a first gas separating tower aftercooler (110), a first gas separating tower phase splitter (111), a second gas separating tower (112), a degassing tank (113), a recovered gas compressor (114), a water washing tower (115), an absorption tower (116), an ethylene recovery tower (117), an acetic acid flash tank (118), an acetic acid recovery tower (119), an acetic acid recovery tower condenser (120), a vacuum unit (121), a desorption tower (122), a desorption tower condenser (123), and a complete set of heating and conveying equipment. A connection relationship is as follows: the circulating gas compressor (101) is connected with an inlet of a heating side of the second reactor outlet heat exchanger (105); an outlet of the heating side of the second reactor outlet heat exchanger (105) is connected with an inlet in a bottom of the acetic acid evaporator (103); an outlet in a top of the acetic acid evaporator (103) is connected with an inlet of a heating side of the first reactor outlet heat exchanger (104); an outlet of the heating side of the first reactor outlet heat exchanger (104) is connected with the circulating ethylene preheater (102); the circulating ethylene preheater (102) is connected with the oxygen mixer (106); an outlet of the oxygen mixer (106) is connected with an inlet of the synthesis reactor (107); an outlet of the synthesis reactor (107) is sequentially connected with cooling sides of the first reactor outlet heat exchanger (104) and the second reactor outlet heat exchanger (105); an outlet of the cooling side of the second reactor outlet heat exchanger (105) is connected with a feed port in a bottom of the first gas separating tower (108); a top of the first gas separating tower (108) is sequentially connected with the first gas separating tower condenser (109) and the first gas separating tower aftercooler (110); the first gas separating tower condenser (109) and the first gas separating tower aftercooler (110) are connected with the first gas separating tower phase splitter (111); a water side of the first gas separating tower phase splitter (111) is fed to a rectifying section, and an oil side is connected with a reflux port of the first gas separating tower (108); a non-condensible gas outlet of the first gas separating tower aftercooler (110) is connected with a feed port in a bottom of the second gas separating tower (112); a top of the second gas separating tower (112) is connected with the circulating gas compressor (101), and a tower kettle outlet of the second gas separating tower (112) is connected with the degassing tank (113); a gas phase outlet of the degassing tank (113) is connected with the recovered gas compressor (114); the recovered gas compressor (114) is connected with an inlet of the water washing tower (115); a tower top outlet of the water washing tower (115) is connected with an inlet in a bottom of the absorption tower (116); an outlet in a top of the absorption tower (116) is connected with the circulating gas compressor (101), an impurity removal outlet and an inlet in a bottom of the ethylene recovery tower (117), and a tower kettle outlet of the ethylene recovery tower (117) is connected with an inlet in a top of the acetic acid evaporator (103); a tower kettle outlet of the acetic acid evaporator (103) is connected with the acetic acid flash tank (118), and an outlet in a bottom of the acetic acid flash tank (118) is connected with a feed port of the acetic acid recovery tower (119); a tower top outlet of the acetic acid recovery tower (119) is connected with the acetic acid recovery tower condenser (120), a condensate outlet of the acetic acid condenser is connected with a reflux port in a tower top of the acetic acid recovery tower (119), and a non-condensible gas outlet of the acetic acid recovery tower condenser (120) is connected with the vacuum unit (121); a liquid phase outlet of the vacuum unit (121) is connected with a feed port of the acetic acid recovery tower (119), and a gas phase outlet of the vacuum unit (121) is connected with an inlet of the degassing tank (113); and a tower kettle of the absorption tower (116) is connected with a tower top inlet of the desorption tower (122), a carbon dioxide stream outlet in a top of the desorption tower (122) is connected with an inlet of the desorption tower condenser (123), and a condensate outlet of the desorption tower condenser (123) is connected with an absorption liquid feed port of the absorption tower (116).

In the above technical solution, an operating pressure of the acetic acid evaporator (103) is 1.0-1.2 bara, and a tower top temperature is 40-100° C.

In the above technical solution, a reaction temperature of the synthesis reactor (107) is 100-180° C., and a reaction pressure is 1.0-1.2 bara; an operating pressure of the first gas separating tower (108) is 6-9 bara, and a tower top temperature is 65-100° C.; and an operating pressure of the second gas separating tower (112) is 6-9 bara, and a tower top temperature is 20-50° C.

In the above technical solution, an operating pressure of the water washing tower (115) is 8-11 bara, and a tower top temperature is 22-55° C.; an operating pressure of the absorption tower (116) is 8-11 bara, and a tower top temperature is 92-112° C.; and an operating pressure of the ethylene recovery tower (117) is 7-8 bara, and a tower top temperature is 23-45° C.

In the above technical solution, an operating pressure of the acetic acid flash tank (118) is 1.0-1.2 bara, and a tower top temperature is 92-115° C.; an operating pressure of the acetic acid recovery tower (119) is 1.0-1.2 bara, and a tower top temperature is 77-91° C.; and an operating pressure of the desorption tower (122) is 1.0-1.3 bara, and a tower top temperature is 103-124° C.

As understood by those skilled in the art, vinyl acetate synthetic reaction liquid also contains acetic acid, water, low-boiling-point components and high-boiling-point components in addition to vinyl acetate. After part of a liquid mixture with a significant difference in boiling points of all components is vaporized at a certain temperature, a composition of a gas phase thereof differs from a composition of a liquid phase, and a proportion of volatile substances in the gas phase is more than that of volatile substances in the liquid phase, so that the liquid mixture can be separated and purified by a rectifying method. Generally, a vinyl acetate rectifying section (vinyl acetate refining process) includes an acetic acid tower, a coarse VAC tower, a fine VAC tower, a de-heavy component tower, an aldehydo-ester concentration tower, an acetaldehyde tower and an acetic acid recovery tower. In vinyl acetate rectifying, all components in the reaction liquid are separated through a series of rectifying operations by means of the difference in relative volatility among the components, and finally vinyl acetate products with high purity and various byproducts are obtained.

The specific implementation process of the method of the present application is described with the specific embodiments below.

### EMBODIMENT 1

Fresh ethylene and circulating gas are mixed, and the mixture is introduced into a circulating gas compressor for heat exchange with reaction gas at a second reactor outlet heat exchanger and then introduced into a bottom of an acetic acid evaporator. Tower bottoms of the ethylene recovery tower are sprayed from a top of the acetic acid evaporator, mixed gas of the ethylene and acetic acid is led out of the top of the evaporator, and the tower bottoms of the evaporator are fed to the acetic acid recovery system. An operating pressure of the acetic acid evaporator is 1.0 bara, and a tower top temperature is 42° C.

After being led out of the top of the acetic acid evaporator, the mixed gas of the ethylene and the acetic acid is heated by a first reactor outlet heat exchanger (104) and a circulating ethylene preheater respectively and then mixed with oxygen by an oxygen mixer. The mixed gas led out of the oxygen mixer is fed into the synthesis reactor from a top. In the embodiment, the circulating gas contains ethane as inert gas, and an ethane concentration at an inlet of a reactor is 9 mol %. An oxygen concentration reaches 6 mol % after the gas is mixed with the oxygen in the oxygen mixer.

After reacting in the synthesis reactor at a reaction temperature of 100° C. and a reaction pressure of 1.0 bara, the reaction gas at an outlet of the reactor is subjected to heat exchange by the first reactor outlet heat exchanger and the second reactor outlet heat exchanger and then fed into a bottom of a first gas separating tower. Dehydrated reaction liquid is obtained at a tower kettle of the first gas separating tower and fed into a rectifying section for refining treatment. Overhead gas mainly consisting of vinyl acetate and water is obtained from a top of the first gas separating tower and fed into the first gas separating tower condenser to be condensed, non-condensible gas of the first gas separating tower condenser is fed into the first gas separating tower aftercooler to be further cooled, condensate of the first gas separating tower condenser and the first gas separating tower aftercooler enters the first gas separating tower phase splitter to be subjected to phase splitting, an oil phase obtained after phase splitting is fed into the first gas separating tower as a reflux, and a water phase is fed into the rectifying section to be further treated. An operating pressure of the first gas separating tower (108) is 6.2 bara, and a tower top temperature is 67° C.

Non-condensible gas cooled by the first gas separating tower aftercooler is fed into a bottom of a second gas separating tower and subjected to absorption separation by the reaction liquid and the acetic acid, then a certain amount of reaction liquid is continuously extracted from a tower kettle and fed to a degassing tank, and gas removed from the degassing tank is compressed by a recovered gas compressor and then fed into a water washing tower. Mixed gas mainly consisting of the ethylene, carbon dioxide, ethane and the oxygen is obtained on a top of the second gas separating tower and fed to the circulating gas compressor as circulating gas. An operating pressure of the second gas separating tower is 6.1 bara, and a tower top temperature is 22° C.

After the gas fed into the water washing tower is washed with water, overhead gas is fed into the absorption tower, so that the carbon dioxide therein is absorbed by alkaline liquid, most of gas led out of a top of the absorption tower is fed to the circulating gas compressor, the rest is fed to an ethylene recovery tower and an impurity removal outlet, and tower bottoms of the absorption tower are fed into a desorption system. An operating pressure of the water washing tower is 8.3 bara, and a tower top temperature is 24° C.

After the overhead gas of the absorption tower is fed into the ethylene recovery tower, fresh acetic acid is added to a top of the ethylene recovery tower to recover the ethylene gas therein, tower bottoms of the ethylene recovery tower are fed to a top of the acetic acid evaporator, and overhead gas of the ethylene recovery tower is fed to be incinerated. An operating pressure of the absorption tower is 8.1 bara, and a tower top temperature is 92° C.; and an operating pressure of the ethylene recovery tower is 7.0 bara, and a tower top temperature is 23° C.

Wherein the acetic acid recovery system includes an acetic acid flash tank, an acetic acid recovery tower, an acetic acid recovery tower condenser and a vacuum unit; tower bottoms of the acetic acid evaporator firstly enter the acetic acid flash tank, gas evaporated from the flash tank is fed to the rectifying section, the tower bottoms of the flash tank are fed to the acetic acid recovery tower, overhead gas of the acetic acid recovery tower is condensed by the acetic acid recovery tower condenser and then refluxes, gas not condensed by the acetic acid recovery tower condenser is fed to the degassing tank after passing through the vacuum unit, and condensate of the vacuum unit is fed to the acetic acid recovery tower. An operating pressure of the acetic acid flash tank is 1.0 bara, and a tower top temperature is 93° C.; and an operating pressure of the acetic acid recovery tower is 1.0 bara, and a tower top temperature is 77° C.

Wherein the desorption system includes a desorption tower and a desorption tower top condenser; and the tower bottoms of the absorption tower are fed to the desorption tower from a top of the desorption tower, two streams of materials are extracted from the top of the desorption tower, one stream is a material mainly containing the ethylene, which is fed to the degassing tank, the other stream mainly consists of the carbon dioxide, after being condensed by the desorption tower top condenser, non-condensible carbon dioxide gas is fed out of a boundary area, and condensed condensate is mixed with tower bottoms of the desorption tower and fed back to the absorption tower along with added fresh alkaline liquid. An operating pressure of the desorption tower is 1.1 bara, and a tower top temperature is 103° C.

In the embodiment, ethane is used as inert gas, an ethane concentration at an inlet of the reactor is 9 mol %, and an oxygen concentration is 6 mol %. A conversion per pass is 27% across the reaction, and selectivity is 96% based on the acetic acid.

### EMBODIMENT 2

Fresh ethylene and circulating gas are mixed, and the mixture is introduced into a circulating gas compressor for heat exchange with reaction gas at a second reactor outlet heat exchanger and then introduced into a bottom of an acetic acid evaporator. Tower bottoms of the ethylene recovery tower are sprayed from a top of the acetic acid evaporator, mixed gas of the ethylene and acetic acid is led out of the top of the evaporator, and the tower bottoms of the evaporator are fed to the acetic acid recovery system. An operating pressure of the acetic acid evaporator is 1.1 bara, and a tower top temperature is 60° C.

After being led out of the top of the acetic acid evaporator, the mixed gas of the ethylene and the acetic acid is heated by a first reactor outlet heat exchanger (104) and a circulating ethylene preheater respectively and then mixed with oxygen by an oxygen mixer. The mixed gas led out of the oxygen mixer is fed into the synthesis reactor from a top. In the embodiment, the circulating gas contains ethane as inert gas, an ethane concentration at an inlet of the reactor is 15 mol %, and an oxygen concentration reaches 10 mol % after the circulating gas is mixed with the oxygen in the oxygen mixer.

After reacting in the synthesis reactor at a reaction temperature of 140° C. and a reaction pressure of 1.2 bara, the reaction gas at an outlet of the reactor is subjected to heat exchange by the first reactor outlet heat exchanger and the second reactor outlet heat exchanger and then fed into a bottom of a first gas separating tower. Dehydrated reaction liquid is obtained at a tower kettle of the first gas separating tower and fed into a rectifying section for refining treatment. Overhead gas mainly consisting of vinyl acetate and water is obtained from a top of the first gas separating tower and fed into the first gas separating tower condenser to be condensed, non-condensible gas of the first gas separating tower condenser is fed into the first gas separating tower aftercooler to be further cooled, condensate of the first gas separating tower condenser and the first gas separating tower aftercooler enters the first gas separating tower phase splitter to be subjected to phase splitting, an oil phase obtained after phase splitting is fed into the first gas separating tower as a reflux, and a water phase is fed into the rectifying section to be further treated. An operating pressure of the first gas separating tower (108) is 8.1 bara, and a tower top temperature is 72° C.

Non-condensible gas cooled by the first gas separating tower aftercooler is fed into a bottom of the second gas separating tower and subjected to absorption separation by the reaction liquid and the acetic acid, then a certain amount of reaction liquid is continuously extracted from a tower kettle and fed to the degassing tank, and gas removed from the degassing tank is compressed by the recovered gas compressor and then fed into the water washing tower. Mixed gas mainly consisting of the ethylene, carbon dioxide, ethane and the oxygen is obtained on a top of the second gas separating tower and fed to the circulating gas compressor as circulating gas. An operating pressure of the second gas separating tower is 8.4 bara, and a tower top temperature is 30° C.

After the gas fed into the water washing tower is washed with water, overhead gas is fed into the absorption tower, so that the carbon dioxide therein is absorbed by alkaline liquid, most of gas led out of a top of the absorption tower is fed to the circulating gas compressor, the rest is fed to an ethylene recovery tower and an impurity removal outlet, and tower bottoms of the absorption tower are fed into a desorption system. An operating pressure of the water washing tower is 8.4 bara, and a tower top temperature is 33° C.

After the overhead gas of the absorption tower is fed into the ethylene recovery tower, fresh acetic acid is added to a top of the ethylene recovery tower to recover the ethylene gas therein, tower bottoms of the ethylene recovery tower are fed to a top of the acetic acid evaporator, and overhead gas of the ethylene recovery tower is fed to be incinerated. An operating pressure of the absorption tower is 8.4 bara, and a tower top temperature is 97° C.; and an operating pressure of the ethylene recovery tower is 7.6 bara, and a tower top temperature is 36° C.

Wherein the acetic acid recovery system includes an acetic acid flash tank, an acetic acid recovery tower, an acetic acid recovery tower condenser and a vacuum unit; tower bottoms of the acetic acid evaporator firstly enter the acetic acid flash tank, gas evaporated from the flash tank is fed to the rectifying section, the tower bottoms of the flash tank are fed to the acetic acid recovery tower, overhead gas of the acetic acid recovery tower is condensed by the acetic acid recovery tower condenser and then refluxes, gas not condensed by the acetic acid recovery tower condenser is fed to the degassing tank after passing through the vacuum unit, and condensate of the vacuum unit is fed to the acetic acid recovery tower. An operating pressure of the acetic acid flash tank is 1.1 bara, and a tower top temperature is 102° C.; and an operating pressure of the acetic acid recovery tower is 1.1 bara, and a tower top temperature is 85° C.

Wherein the desorption system includes a desorption tower and a desorption tower top condenser; and the tower bottoms of the absorption tower are fed to the desorption tower from a top of the desorption tower, two streams of materials are extracted from the top of the desorption tower, one stream is a material mainly containing the ethylene, which is fed to the degassing tank, the other stream mainly consists of the carbon dioxide, after being condensed by the desorption tower top condenser, non-condensible carbon dioxide gas is fed out of a boundary area, and condensed condensate is mixed with tower bottoms of the desorption tower and fed back to the absorption tower along with added fresh alkaline liquid. An operating pressure of the desorption tower is 1.2 bara, and a tower top temperature is 119° C.

In the embodiment, ethane is used as inert gas, an ethane concentration at an inlet of the reactor is 15 mol %, and an oxygen concentration is 10 mol %. A conversion per pass is 35% across the reaction, and selectivity is 99% based on the acetic acid.

### EMBODIMENT 3

Fresh ethylene and circulating gas are mixed, and the mixture is introduced into a circulating gas compressor for heat exchange with reaction gas at a second reactor outlet heat exchanger and then introduced into a bottom of an acetic acid evaporator. Tower bottoms of the ethylene recovery tower are sprayed from a top of the acetic acid evaporator, mixed gas of the ethylene and acetic acid is led out of the top of the evaporator, and the tower bottoms of the evaporator are fed to the acetic acid recovery system. An operating pressure of the acetic acid evaporator is 1.2 bara, and a tower top temperature is 98° C.

After being led out of the top of the acetic acid evaporator, the mixed gas of the ethylene and the acetic acid is heated by a first reactor outlet heat exchanger (104) and a circulating ethylene preheater respectively and then mixed with oxygen by an oxygen mixer. The mixed gas led out of the oxygen mixer is fed into the synthesis reactor from a top. In the embodiment, the circulating gas contains ethane as inert gas, and an ethane concentration at an inlet of a reactor is 18 mol %. The oxygen concentration reaches 12 mol % after the circulating gas is mixed with the oxygen in the oxygen mixer.

After reacting in the synthesis reactor at a reaction temperature of 180° C. and a reaction pressure of 1.2 bara, the reaction gas at an outlet of the reactor is subjected to heat exchange by the first reactor outlet heat exchanger and the second reactor outlet heat exchanger and then fed into a bottom of a first gas separating tower. Dehydrated reaction liquid is obtained at a tower kettle of the first gas separating tower and fed into a rectifying section for refining treatment. Overhead gas mainly consisting of vinyl acetate and water is obtained from a top of the first gas separating tower and fed into the first gas separating tower condenser to be condensed, non-condensible gas of the first gas separating tower condenser is fed into the first gas separating tower aftercooler to be further cooled, condensate of the first gas separating tower condenser and the first gas separating tower aftercooler enters the first gas separating tower phase splitter to be subjected to phase splitting, an oil phase obtained after phase splitting is fed into the first gas separating tower as a reflux, and a water phase is fed into the rectifying section to be further treated. An operating pressure of the first gas separating tower (108) is 9 bara, and a tower top temperature is 97° C.

Non-condensible gas cooled by the first gas separating tower aftercooler is fed into a bottom of the second gas separating tower and subjected to absorption separation by the reaction liquid and the acetic acid, then a certain amount of reaction liquid is continuously extracted from a tower kettle and fed to the degassing tank, and gas removed from the degassing tank is compressed by the recovered gas compressor and then fed into the water washing tower. Mixed gas mainly consisting of the ethylene, carbon dioxide, ethane and the oxygen is obtained on a top of the second gas separating tower and fed to the circulating gas compressor as circulating gas. An operating pressure of the second gas separating tower is 8.8 bara, and a tower top temperature is 47° C.

After the gas fed into the water washing tower is washed with water, overhead gas is fed into the absorption tower, so that the carbon dioxide therein is absorbed by alkaline liquid, most of gas led out of a top of the absorption tower is fed to the circulating gas compressor, the rest is fed to an ethylene recovery tower and an impurity removal outlet, and tower bottoms of the absorption tower are fed into a desorption system. An operating pressure of the water washing tower is 11 bara, and a tower top temperature is 55° C.

After the overhead gas of the absorption tower is fed into the ethylene recovery tower, fresh acetic acid is added to a top of the ethylene recovery tower to recover the ethylene gas therein, tower bottoms of the ethylene recovery tower are fed to a top of the acetic acid evaporator, and overhead gas of the ethylene recovery tower is fed to be incinerated. An operating pressure of the absorption tower is 11 bara, and a tower top temperature is 110° C.; and an operating pressure of the ethylene recovery tower is 8 bara, and a tower top temperature is 45° C.

Wherein the acetic acid recovery system includes an acetic acid flash tank, an acetic acid recovery tower, an acetic acid recovery tower condenser and a vacuum unit; tower bottoms of the acetic acid evaporator firstly enter the acetic acid flash tank, gas evaporated from the flash tank is fed to the rectifying section, the tower bottoms of the flash tank are fed to the acetic acid recovery tower, overhead gas of the acetic acid recovery tower is condensed by the acetic acid recovery tower condenser and then refluxes, gas not condensed by the acetic acid recovery tower condenser is fed to the degassing tank after passing through the vacuum unit, and condensate of the vacuum unit is fed to the acetic acid recovery tower. An operating pressure of the acetic acid flash tank is 1.2 bara, and a tower top temperature is 115° C.; and an operating pressure of the acetic acid recovery tower is 1.2 bara, and a tower top temperature is 90° C.

Wherein the desorption system includes a desorption tower and a desorption tower top condenser; and the tower bottoms of the absorption tower are fed to the desorption tower from a top of the desorption tower, two streams of materials are extracted from the top of the desorption tower, one stream is a material mainly containing the ethylene, which is fed to the degassing tank, the other stream mainly consists of the carbon dioxide, after being condensed by the desorption tower top condenser, non-condensible carbon dioxide gas is fed out of a boundary area, and condensed condensate is mixed with tower bottoms of the desorption tower and fed back to the absorption tower along with added fresh alkaline liquid. An operating pressure of the desorption tower is 1.3 bara, and a tower top temperature is 124° C.

In the embodiment, ethane is used as inert gas, an ethane concentration at an inlet of the reactor is 18 mol %, and an oxygen concentration is 12 mol %. A conversion per pass is 33% across the reaction, and selectivity is 91% based on the acetic acid.

The technical solutions disclosed and provided by the present disclosure can be achieved by properly changing conditions, routes and other links by those skilled in the art by referring to the content herein. Although the method and preparation technology of the present disclosure have been described through the preferred embodiments, related technicians obviously can change or recombine the method and technical route herein without departing from the content, spirit and scope of the present disclosure, so as to achieve the final preparation technology. It should be particularly noted that all the similar replacements and changes are apparent to those skilled in the art, and they are all deemed to be included in the spirit, scope and content of the present disclosure.

1. A production process of vinyl acetate, comprising a circulating gas compressor, an acetic acid evaporator, a circulating ethylene preheater, an oxygen mixer, a synthesis reactor, a first reactor outlet heat exchanger, a second reactor outlet heat exchanger, a first gas separating tower, a first gas separating tower condenser, a first gas separating tower aftercooler, a first gas separating tower phase splitter, a second gas separating tower, a degassing tank, a recovered gas compressor, a water washing tower, an absorption tower, an ethylene recovery tower, an acetic acid recovery system and a desorption system; wherein
(1) mixing fresh ethylene and circulating gas, introducing the mixture into the circulating gas compressor, and introducing the mixture into a bottom of the acetic acid evaporator after performing heat exchange with streams at an outlet of the reactor by the second reactor outlet heat exchanger; and spraying tower bottoms of the ethylene recovery tower from a top of the acetic acid evaporator, leading mixed gas of the ethylene and acetic acid out of the top of the evaporator, and feeding the tower bottoms of the evaporator to the acetic acid recovery system;
(2) after leading the mixed gas of the ethylene and the acetic acid out of the top of the acetic acid evaporator, heating the mixed gas by the first reactor outlet heat exchanger and the circulating ethylene preheater respectively, and then mixing the mixed gas with oxygen by the oxygen mixer; and feeding the mixed gas from the oxygen mixer into the synthesis reactor from a top;
(3) performing heat exchange on reaction gas at the outlet of the reactor by the first reactor outlet heat exchanger and the second reactor outlet heat exchanger respectively, feeding the reaction gas into a bottom of the first gas separating tower; and obtaining dehydrated reaction liquid at a tower kettle of the first gas separating tower, and feeding it to a rectifying section for refining treatment; obtaining overhead gas mainly consisting of vinyl acetate and water from a top of the first gas separating tower, feeding it into the first gas separating tower condenser to be condensed, feeding non-condensible gas of the first gas separating tower condenser into the first gas separating tower aftercooler to be further cooled, making condensate of the first gas separating tower condenser and the first gas separating tower aftercooler enter the first gas separating tower phase splitter to be subjected to phase splitting, feeding an oil phase obtained after phase splitting into the first gas separating tower as a reflux, and feeding a water phase into the rectifying section to be further treated;
(4) feeding non-condensible gas cooled by the first gas separating tower aftercooler into a bottom of the second gas separating tower, continuously extracting a certain amount of reaction liquid from a tower kettle after absorption separation by the reaction liquid and the acetic acid, feeding the reaction liquid to the degassing tank, compressing gas removed from the degassing tank by the recovered gas compressor, and then feeding the gas into the water washing tower; and obtaining mixed gas mainly consisting of the ethylene, carbon dioxide, ethane and the oxygen on a top of the second gas separating tower, and feeding the mixed gas to the circulating gas compressor as circulating gas;
(5) after washing the gas fed into the water washing tower with water, feeding overhead gas into the absorption tower for the absorption of the carbon dioxide therein by alkaline liquid, feeding most of gas led out of a top of the absorption tower to the circulating gas compressor, feeding the rest to the ethylene recovery tower and an impurity removal outlet, and feeding tower bottoms of the absorption tower to the desorption system; and
(6) After the overhead gas of the absorption tower is fed into the ethylene recovery tower, fresh acetic acid is added to a top of the ethylene recovery tower to recover the ethylene gas therein, and the tower bottoms of the ethylene recovery tower are fed to the tower top of the acetic acid evaporator and the overhead gas of the ethylene recovery tower is fed to be incinerated.

2. The production process of the vinyl acetate according to claim 1, wherein the acetic acid recovery system comprises an acetic acid flash tank, an acetic acid recovery tower, an acetic acid recovery tower condenser and a vacuum unit; and a method comprises the steps of feeding tower bottoms of the acetic acid evaporator firstly into the acetic acid flash tank, feeding gas evaporated from the flash tank to the rectifying section, feeding the tower bottoms of the flash tank to the acetic acid recovery tower, condensing overhead gas of the acetic acid recovery tower by the acetic acid recovery tower condenser and then refluxing, feeding gas not condensed by the acetic acid recovery tower condenser to the degassing tank after passing through the vacuum unit, and feeding condensate of the vacuum unit to the acetic acid recovery tower.

3. The production process of the vinyl acetate according to claim 1, wherein the desorption system comprises a desorption tower and a desorption tower top condenser; and a method comprises the steps of feeding the tower bottoms of the absorption tower into the desorption tower from a top of the desorption tower, extracting two streams of materials from the top of the desorption tower, one stream being a material containing the ethylene, which is fed to the degassing tank, the other stream mainly consisting of the carbon dioxide, after being condensed by the desorption tower top condenser, feeding non-condensible carbon dioxide gas out of a boundary area, and mixing condensed condensate mixed with the tower bottoms of the desorption tower and feeding back to the absorption tower along with added fresh alkaline liquid.

4. The production process of the vinyl acetate according to claim 1, wherein the circulating gas contains ethane gas, and a concentration of the ethane gas at an inlet of the reactor is 9-18 mol %.

5. The production process of the vinyl acetate according to claim 1, wherein an oxygen concentration at the inlet of the reactor is 6-12 mol %.

6. A production device of vinyl acetate, further comprising a circulating gas compressor (101), a circulating ethylene preheater (102), an acetic acid evaporator (103), a first reactor outlet heat exchanger (104), a second reactor outlet heat exchanger (105), an oxygen mixer (106), a synthesis reactor (107), a first gas separating tower (108), a first gas separating tower condenser (109), a first gas separating tower aftercooler (110), a first gas separating tower phase splitter (111), a second gas separating tower (112), a degassing tank (113), a recovered gas compressor (114), a water washing tower (115), an absorption tower (116), an ethylene recovery tower (117), an acetic acid flash tank (118), an acetic acid recovery tower (119), an acetic acid recovery tower condenser (120), a vacuum unit (121), a desorption tower (122), a desorption tower condenser (123), and a complete set of heating and conveying equipment; and a connection relationship is as follows: the circulating gas compressor (101) is connected with an inlet of a heating side of the second reactor outlet heat exchanger (105); an outlet of the heating side of the second reactor outlet heat exchanger (105) is connected with an inlet in a bottom of the acetic acid evaporator (103); an outlet in a top of the acetic acid evaporator (103) is connected with an inlet of a heating side of the first reactor outlet heat exchanger (104); an outlet of the heating side of the first reactor outlet heat exchanger (104) is connected with the circulating ethylene preheater (102); the circulating ethylene preheater (102) is connected with the oxygen mixer (106); an outlet of the oxygen mixer (106) is connected with an inlet of the synthesis reactor (107); an outlet of the synthesis reactor (107) is sequentially connected with cooling sides of the first reactor outlet heat exchanger (104) and the second reactor outlet heat exchanger (105); an outlet of the cooling side of the second reactor outlet heat exchanger (105) is connected with a feed port in a bottom of the first gas separating tower (108); a top of the first gas separating tower (108) is sequentially connected with the first gas separating tower condenser (109) and the first gas separating tower aftercooler (110); the first gas separating tower condenser (109) and the first gas separating tower aftercooler (110) are connected with the first gas separating tower phase splitter (111); a water side of the first gas separating tower phase splitter (111) is fed to a rectifying section, and an oil side is connected with a reflux port of the first gas separating tower (108); a non-condensible gas outlet of the first gas separating tower aftercooler (110) is connected with a feed port in a bottom of the second gas separating tower (112); a top of the second gas separating tower (112) is connected with the circulating gas compressor (101), and a tower kettle outlet of the second gas separating tower (112) is connected with the degassing tank (113); a gas phase outlet of the degassing tank (113) is connected with the recovered gas compressor (114); the recovered gas compressor (114) is connected with an inlet of the water washing tower (115); a tower top outlet of the water washing tower (115) is connected with an inlet in a bottom of the absorption tower (116); an outlet in a top of the absorption tower (116) is connected with the circulating gas compressor (101), an impurity removal outlet and an inlet in a bottom of the ethylene recovery tower (117), and a tower kettle outlet of the ethylene recovery tower (117) is connected with an inlet in a top of the acetic acid evaporator (103); a tower kettle outlet of the acetic acid evaporator (103) is connected with the acetic acid flash tank (118), and an outlet in a bottom of the acetic acid flash tank (118) is connected with a feed port of the acetic acid recovery tower (119); a tower top outlet of the acetic acid recovery tower (119) is connected with the acetic acid recovery tower condenser (120), a condensate outlet of the acetic acid condenser is connected with a reflux port in a tower top of the acetic acid recovery tower (119), and a non-condensible gas outlet of the acetic acid recovery tower condenser (120) is connected with the vacuum unit (121); a liquid phase outlet of the vacuum unit (121) is connected with a feed port of the acetic acid recovery tower (119), and a gas phase outlet of the vacuum unit (121) is connected with an inlet of the degassing tank (113); and a tower kettle of the absorption tower (116) is connected with a tower top inlet of the desorption tower (122), a carbon dioxide stream outlet in a top of the desorption tower (122) is connected with an inlet of the desorption tower condenser (123), and a condensate outlet of the desorption tower condenser (123) is connected with an absorption liquid feed port of the absorption tower (116).

7. The production device according to claim 6, wherein an operating pressure of the acetic acid evaporator (103) is 1.0-1.2 bara, and a tower top temperature is 40-100° C.

8. The production device according to claim 6, wherein a reaction temperature of the synthesis reactor (107) is 100-180° C., and a reaction pressure is 1.0-1.2 bara; an operating pressure of the first gas separating tower (108) is 6-9 bara, and a tower top temperature is 65-100° C.; and an operating pressure of the second gas separating tower (112) is 6-9 bara, and a tower top temperature is 20-50° C.

9. The production device according to claim 6, wherein an operating pressure of the water washing tower (115) is 8-11 bara, and a tower top temperature is 22-55° C.; an operating pressure of the absorption tower (116) is 8-11 bara, and a tower top temperature is 92-112° C.; and an operating pressure of the ethylene recovery tower (117) is 7-8 bara, and a tower top temperature is 23-45° C.

10. The production device according to claim 6, wherein an operating pressure of the acetic acid flash tank (118) is 1.0-1.2 bara, and a tower top temperature is 92-115° C.; an operating pressure of the acetic acid recovery tower (119) is 1.0-1.2 bara, and a tower top temperature is 77-91° C.; and an operating pressure of the desorption tower (122) is 1.0-1.3 bara, and a tower top temperature is 103-124° C.
